(19) United States
(12) Patent Application Publication
(10) Pub. No.: US 2007/0166411 A1
(43) Pub. Date: Jul. 19, 2007

(54) Nutritional supplement containing long-chain polyunsaturated fatty acids

(75) Inventors: Joshua C. Anthony, Evansville, IN (US); Catherine Peterson, Columbia, MO (US); Kevin A. Sims, Evansville, IN (US); Deborah Schade, Evansville, IN (US); Monjur Hossen, Battle Creek, MI (US); Priscilla Samuel, Evansville, IN (US)

Correspondence Address: BRISTOL-MYERS SQUIBB COMPANY - MEAD JOHNSON, 2400 WEST LLOYD EXPRESSWAY, PATENT DEPARTMENT, EVANSVILLE, IN, 47721, US

(73) Assignee: BRISTOL-MYERS SQUIBB COMPANY, PRINCETON, NJ (US)

(21) Appl. No.: 11/637,484

(22) Filed: Dec. 11, 2006

Related U.S. Application Data
(63) Continuation-in-part of application No. 11/303,374, filed on Dec. 16, 2005, now abandoned.

(51) Int. Cl. A61K 36/899; A61K 36/28; A61K 36/31; A01N 37/06; A61K 31/724; A61K 31/7012; A61K 31/22

(52) U.S. Cl. 424/750; 424/755; 424/757; 424/764; 514/2; 514/547; 514/560; 514/53; 514/58

(57) ABSTRACT

The present invention relates to a nutritional supplement for administration to children. The supplement comprises a protein component, a carbohydrate component, and a fat or lipid component which further comprises a source of DHA. The supplement has an ω-6:ω3 fatty acid ratio of about 6:1 or less. The present invention also relates to a method of providing nutrition to a pediatric subject comprising administering to the subject a nutritional supplement comprising a protein component; a carbohydrate component; and a fat or lipid component, which further comprises a source of DHA, wherein the composition has an ω-6:ω-3 fatty acid ratio of about 6:1 or less.

### CROSS-REFERENCE TO RELATED PATENTS AND PATENT APPLICATIONS

This application is a continuation-in-part application and claims the priority benefit of U.S. patent application Ser. No. 11/303,374, filed Dec. 16, 2005.

### BACKGROUND OF THE INVENTION

(1) Field of the Invention

The present invention relates to a nutritional supplement, as a meal replacement or snack, or as a nutrient for subjects suffering from chronic illness, trauma, or failure to thrive that contains a unique lipid blend including a level of DHA supplementation and a low ω-6:ω-3 fatty acid ratio relative to the typical western diet in order to provide health benefits and/or to prevent adverse health consequences.

(2) Description of the Related Art

Toddlers and children in developed countries are notoriously picky eaters. These eating habits coupled with poor parental food selection can often result in children not receiving adequate levels of key nutrients important to optimize growth and development. One such nutrient is the ω-3 long-chain polyunsaturated fatty acid (LCPUFA), docosahexaenoic acid (DHA).

DHA has been shown to be important for cognitive and visual development and accumulates rapidly in the developing brain for at least the first two years of life. To date, there is no nutritionally complete product intended for children ages 1-12 that contains an appropriate level of DHA supplementation. This is due in part to limited information regarding LCPUFA intake of weaning infants and toddlers or potential benefits from the consumption of a diet including ω-3 LCPUFA in this age group.

However, a recent survey of dietary intakes in U.S. children 1-12 years of age reported intakes of DHA are 30 mg/day or less. Extrapolation of existing recommendations for infants or adults to children in this age group suggests that DHA intakes of 40-179 mg/day may be desired to provide levels that have been shown to promote health benefits, including optimal brain and cardiovascular health and function in other age groups.

These results suggest that a “gap” of up to 150 mg/day may exist between the typical level of DHA intake in young children and what might be considered a desirable level of supplementation. Further, depending on the specific benefit, efficacy-based levels may be as high as 500 mg/day for some of these age groups. This is particularly important when one considers that recommended DHA levels in newborns and adults are designed to optimize brain and cardiovascular health and function. To date, however, no products have been brought to the market that are specifically designed to address this gap in DHA intake for children ages 1-12.

A nutritionally complete product supplemented with an appropriate level of DHA, in combination with an appropriate blend of dietary oils, may also help to address other growing health concerns. One of those concerns is increasing the level of ω-6 fatty acids relative to ω-3 fatty acids in the diet.

Currently, western diets contain a ratio of ω-6:ω-3 fatty acids of more than 15:1. This change in fatty acid consumption toward excessive intakes of ω-6 polyunsaturated fatty acids (PUFAs) resulting in a very high ω-6:ω-3 ratio has been implicated in the pathogenesis of a variety of diseases including cardiovascular, cancer, inflammatory, and autoimmune diseases. In children, a greater number of behavior problems, temper tantrums, sleep problems, learning detriments, and health problems have been reported in subjects with lower total ω-3 fatty acid concentrations, underscoring the need for adequate ω-3 fatty acid nutrition in children.

There is a growing body of scientific evidence indicating the health benefits of a lower ω-6:ω-3 fatty acid ratio, including immune function, cardiovascular, bone, and mental health benefits. In particular, studies suggest that an ω-6:ω-3 ratio of less than 6:1 may be associated with health benefits, whereas a ratio of 10:1 or greater may be associated with adverse health effects. Various federal agencies and scientific organizations are placing an increased emphasis on increasing ω-3 fats in the diet.

Available nutritionally complete children's products are currently used for supplemental nutrition, a meal replacement or snack, or for chronic illness, injury, trauma, or failure to thrive in young children. These products are generally formulas that focus on providing complete nutrition in pleasant tasting formulations to help promote patient acceptance and compliance. For example, Enfamil® Kindercal® (Mead Johnson, Evansville, Ind.), is intended to provide nutrition for children ages 1 to 10 years of age who require supplemental nutrition, a meal replacement, or a snack, or complete nutrition for chronic illness, injury, or failure to thrive. The currently marketed Kindercal® supplement has an ω-6:ω-3 ratio of about 5:1, but does not contain DHA. Pediasure® (Ross Products Division, Abbott Laboratories, Columbus, Ohio) is designed for children 1 to 10 years of age whose nutrient needs are increased or who may be undernourished due to illness or poor appetite. Likewise, Pediasure® does not contain DHA. However, Pediasure® has an ω-6:ω-3 fatty acid ratio of approximately 10.8:1.

While each of these products is designed to supplement the diet of children who are picky eaters or to sustain the chronically ill pediatric patient, opportunities still exist to improve upon the composition of these formulas by optimizing the lipid composition to provide an evidence-based level of DHA in combination with an appropriate oil blend to maintain a low ω-6:ω-3 fatty acid ratio in order to optimize brain development and function and cardiovascular health.

Because of the problems associated with current nutritional products, improvements have been sought in several reported nutritional supplements. These supplements, however, do not adequately address all of the health problems that arise from diets that are lacking in LCPUFAs and/or have high ratios of ω-6 fatty acids relative to ω-3 fatty acids.

For example, W02003043445 to Cary, et al., discloses a processed baby food useful for supplementing the diet of a child with precursors for the synthesis of arachidonic acid and DHA comprising a blend of linoleic acid and alpha-linolenic acid. Because this supplement only incorporates the precursors of LCPUFAs, it, therefore, lacks DHA. The described invention also specifies a relatively high ω-6 fatty acid:ω-3 fatty acid ratio of 7:1 to 12:1.

W0200215719 to Fuchs, et al., discloses a composition for a nutritional supplement for a sick patient with a lipid source providing at least 18% of the total calories. The lipid source has an ω-6:ω-3 fatty acid ratio of approximately 1:1 to 10:1. However, the supplement lacks DHA.

U.S. patent app. Ser. Nos. 20030000391 and 20030185941 to Highman, et al., disclose an organic nutritional beverage containing at least one of eicosapentaenoic acid (EPA) and DHA. Arachidonic and linolenic acids (ω-6 fatty acids) are also useful fatty acids in the beverage according to the invention. The applications do not disclose any particular ratio of ω-6:ω-3 fatty acids that are present in the beverage.

W02001047377 to Myhre discloses a nutritional liquid preparation containing oil mixed with an aqueous phase in order to improve freshness of the product. The nutritional liquid also has a high ratio of ω-6:ω-3 fatty acids.

U.S. Pat. No. 5,902,797 to Bell, et al., discloses a nutritional supplement containing carbohydrate, protein, and fat, useful for an individual with suppressed appetite. DHA is not disclosed as a part of the supplement.

From the foregoing, it can be seen that a need exists for improved nutritional supplements that contain protein, carbohydrate and lipid sources that include a meaningful level of DHA, yet have lower overall ratios of ω-6 fatty acids to ω-3 fatty acids. Likewise, nutritional compositions that provide health benefits and/or prevent adverse health consequences in children would also be desirable. Finally, compositions that are capable of providing the special nutritional requirements of children that suffer from chronic illness, injury, trauma, or failure to thrive would also be desirable.

### SUMMARY OF THE INVENTION

Briefly, the invention is directed to a novel nutritional composition comprising a protein component; a carbohydrate component; and a fat or lipid component, which further comprises a source of DHA, wherein the supplement has an ω-6:ω-3 fatty acid ratio of about 6:1 or less.

Another aspect of the invention relates to a method of providing nutrition to a subject comprising administering to the subject a nutritional supplement comprising a protein component; a carbohydrate component; and a fat or lipid component, which further comprises a source of DHA, wherein the supplement has an ω-6:ω-3 fatty acid ratio of about 6:1 or less.

In some embodiments, the nutritional supplement is a nutritionally complete supplement. For example, the nutritional supplement may be suitable as the exclusive source of caloric intake for the subject, or it may be optionally complemented with animal milk, human milk and/or other infant milks initially, and/or with solid foods or beverages later.

The lipid blend of the present invention offers an improvement in the ω-6 to ω-3 fatty acid ratio in comparison to conventional nutritional supplements and current nutritionally complete supplements. For example, the invention disclosed herein, in some embodiments, contains a unique lipid blend including a DHA-rich oil source, canola oil, high oleic sunflower oil, corn oil, soy oil and medium-chain triglyceride (MCT) oil that provides an appropriate level of DHA supplementation, and a low ω-6:ω-3 fatty acid ratio relative to the typical western diet in order to provide health benefits and/or to prevent adverse health consequences.

Therefore, the present invention provides an improved nutritional supplement that comprises protein, carbohydrate and lipid sources that include a meaningful level of DHA, yet has lower overall ratios of ω-6 fatty acids to ω-3 fatty acids. Likewise, the present invention provides a nutritional supplement that provides health benefits and/or prevents adverse health consequences in children. Finally, the present invention provides methods and compositions that are capable of supplying the special nutritional requirements of children that suffer from chronic illness, injury, trauma, or failure to thrive.

### DETAILED DESCRIPTION OF SEVERAL EMBODIMENTS

Reference now will be made in detail to the embodiments of the invention, one or more examples of which are set forth below. Each example is provided by way of explanation of the invention, not limitation of the invention. In fact, it will be apparent to those skilled in the art that various modifications and variations can be made in the present invention without departing from the scope or spirit of the invention. For instance, features illustrated or described as part of one embodiment can be used on another embodiment to yield a still further embodiment. Thus, it is intended that the present invention cover such modifications and variations as come within the scope of the appended claims and their equivalents. Other objects, features and aspects of the present invention are disclosed in or are obvious from the following detailed description. It is to be understood by one of ordinary skill in the art that the present discussion is a description of exemplary embodiments only, and is not intended as limiting the broader aspects of the present invention.

In accordance with the present invention, it has been discovered that a pediatric nutritional supplement can be provided in a ready-to-use nutritionally complete form by admixing proteins, lipids, and carbohydrates into a composition in proportions that are appropriate for children. For example, in one embodiment, the present invention provides a nutritional supplement comprising a protein component, a carbohydrate component, and a lipid component that comprises a source of DHA, wherein the supplement has an ω-6:ω-3 fatty acid ratio of about 6:1 or less.

In some embodiments, the nutritional supplement is a nutritionally complete pediatric supplement. As used herein, the terms “pediatric” and “children” refer to human subjects that are between the ages of 9 months and thirteen years old, and in some embodiments, more specifically to children between the ages of one and twelve years old. In a particular embodiment, the children are between the ages of one and ten years old.

As used herein, the term “nutritionally complete” refers to a nutritional supplement that may be used as the sole source of nutrition, which would supply to a subject essentially all the required daily amounts of vitamins, minerals and/or trace elements in combination with the proteins, carbohydrates and lipids.

Advantageously, the present invention provides a benefit over conventional nutritional supplements in that the combination of the lipid blend with a meaningful level of DHA results in an ω-6:ω-3 fatty acid ratio of about 6:1 or less. For example, the ω-6:ω-3 ratio of such conventional products as PediaSure® is approximately 11:1. While not intended to be limiting, it is believed that nutritional intake of lower overall ratios of ω-6 to ω-3 fatty acids may provide certain health benefits, including, but not limited to, improved immune function, cardiovascular improvement, prevention or treatment of asthma and other respiratory illnesses, bone strength, and various mental health benefits.

Though not wishing to be bound to this or any theory, it is believed that the present invention may have additional benefits over convention nutritional supplements as it relates to its effect on intracellcular phosphatidylserine (PS) concentrations. PS is a phospholipid that is essential to the functioning of all the cells of the body, but is particularly concentrated in the brain. PS has been shown to have particular benefits in terms of mind and memory enhancement. It is believed that DHA can modulate PS levels in vivo and in vitro, as it has been shown that PS levels directly correlate with DHA content in neuronal cells. Akbar, Mohammed, et al., Docosahexaenoic Acid: A Positive Modulator of Akt Signaling in Neuronal Survival, PNAS 102(31):10858-10863 (August 2005). Considering the well-established notion that changes in the phospholipid proportion are not easily introduced, modulation of PS by DHA appears to be a unique mechanism. Id.

Trace amounts of PS can be found in lecithin. In certain embodiments of the invention, the nutritional supplement contains both lecithin and DHA. In this combination, it is likely that DHA increases the intracellular concentration of PS. As such, the present invention may be especially helpful in improving brain function, memory, verbal ability, mood, sociability, stress, attention deficit disorders, and/or hyperactive disorders.

The nutrient profile of the present invention may be, in one embodiment, designed primarily for the pediatric subject who cannot or will not consume adequate nutrition through traditional dietary sources. For example, the nutritional supplement can be used as a pediatric nutritional supplement for children who are “picky-eaters”, as a meal replacement (e.g., nutritionally complete) or snack, or for providing the special nutritional requirements of children that suffer from chronic illness, injury, trauma, or failure to thrive. Likewise, the nutritional supplement described herein can be used as a nutritional supplement for convalescing children recovering from illness or surgery, those with limited appetite, anorexic children, bulimic children and for those children which have impaired ability to digest other sources of nutrition.

The nutritional supplement may further comprise vitamins and minerals in amounts sufficient to supply the daily nutritional requirements of children between one and thirteen years old, and in some embodiments, the amounts may be selected according to FDA guidelines. The nutritional supplement may also contain other ingredients such as antioxidants, emulsifiers, stabilizers, preservatives, fiber, coloring agents, flavoring agents such as sweeteners, and other dietary supplements.

In one embodiment, the present invention also encompasses a method of providing nutrition to a subject comprising administering to the subject a nutritional supplement comprising a protein component, a carbohydrate component, and a lipid component that comprises a source of DHA, wherein the nutritional supplement has an ω-6:ω-3 fatty acid ratio of about 6:1 or less. In some embodiments, the subject is a pediatric subject.

Suitable carbohydrates, lipids and proteins can vary widely and are well known to those skilled in the art of making pediatric nutritional supplements. Selection of one or more of the ingredients described herein can be a matter of formulation design or consumer and end-user preferences. For purposes of expediency, several ingredients that are suitable for use with methods and compositions of the present invention are described in further detail below. However, the present invention should not be construed as being limited to any one of the specific ingredients listed herein, since one of skill in the art will readily understand that many suitable ingredients other than the ones described herein could be used in addition to or in lieu thereof.

One component of the methods and compositions of the present invention is one or more carbohydrates. As used herein, the term “carbohydrate” or “carbohydrates” refers to both simple carbohydrates (i.e., monosaccharides and disaccharides) and complex carbohydrates (i.e., polysaccharides).

The nutritional supplement of the present invention encompasses any source of carbohydrates that may be naturally-occurring, synthetic, or developed through the genetic manipulation of organisms, whether such new source is now known or later developed. For example, sources of carbohydrates that are suitable for use with the methods and compositions of the present invention can include, but are not limited to, corn syrup solids; maltodextrin; sugars such as glucose, fructose, dextrose, lactose, galactose, saccharides, sucrose, and maltose; sugar alcohols such as sorbitol, mannitol and xylitol; syrups such as maltitol, corn syrup, rice syrup and high fructose corn syrup; and mixtures thereof.

Commercial sources for the carbohydrates listed above are readily available and known to one practicing the art. For example, corn syrup solids are available from Cerestar USA, Inc. in Hammond, Ind. Glucose and rice based syrups are available from California Natural Products in Lathrop, Calif. Various corn syrups and high fructose corn syrups are available from Cargil in Minneapolis, Minn. Fructose is available from A. E. Staley in Decatur, Ill.

In addition to the carbohydrates described above, the nutritional supplement described herein can also contain artificial sweeteners, e.g., sucralose, saccharine, cyclamates, aspartamine, aspartame, acesulfame K and/or sorbitol. Such artificial sweeteners can be desirable if the nutritional supplement is intended for an overweight subject, or a subject with type 11 diabetes who is prone to hyperglycemia. The nutritional supplement of the present invention encompasses any source of artificial sweeteners that may be naturally-occurring, synthetic, or developed through the genetic manipulation of organisms, whether such new source is now known or later developed.

Another component of the methods and compositions of the present invention is one or more proteins. As used herein, the term “protein” or “proteins” refers to organic compounds that comprise amino acids joined by peptide bonds.

The nutritional supplement of the present invention encompasses any source of protein that may be naturally-occurring, synthetic, or developed through the genetic manipulation of organisms, whether such new source is now known or later developed. For example, sources of proteins that are suitable for use with the methods and compositions of the present invention can include, but are not limited to any suitable protein utilized in nutritional formulations including milk protein, milk protein concentrate, whey protein, whey protein concentrate, whey powder, lactalbumen, egg proteins (e.g., albumin), soy protein, soy protein isolate, rice protein, beef collagen, pea protein, potato protein, casein, caseinate (e.g., sodium caseinate, sodium calcium caseinate, calcium caseinate, potassium caseinate), animal (e.g., beef and fish), and mixtures thereof.

Proteins that are suitable for use with the methods and compositions of the present invention can also include hydrolyzed proteins. As used herein, the term “hydrolyzed proteins” or “protein hydrolysates” means protein which has been processed or treated in a manner intended to break one or more peptide (amide) bonds. Such hydrolyzed peptide fragments and free amino acids are more easily digested. The breakage of amide bonds may occur unintentionally or incidentally during manufacture, for example due to heating or shear.

Suitable protein hydrolysates include, but are not limited to, soy protein hydrolysate, casein protein hydrolysate, whey protein hydrolysate, rice protein hydrolysate, potato protein hydrolysate, fish protein hydrolysate, egg albumen hydrolysate, gelatin protein hydrolysate, a combination of animal and vegetable protein hydrolysates, and mixtures thereof.

In some embodiments, the protein may also be provided in the form of free amino acids. Free amino acids may be added to the supplement in addition to the protein component. Examples of suitable free amino acids include, but are not limited to, histidine, isoleucine, leucine, lysine, methionine, cysteine, phenylalanine, tyrosine, threonine, tryptophan, valine, alanine, arginine, asparagine, aspartic acid, glutamic acid, glutamine, glycine, proline, serine, carnitine, taurine and mixtures thereof. In other embodiments, small amino acid peptides may be included as the protein of the present invention. Such small amino acid peptides may be naturally occurring or synthesized.

In certain aspects of the present invention, the protein component comprises a milk protein concentrate, which is a blend of casein and whey protein. In some embodiments, the protein component comprises a blend of casein and whey, wherein the ratio of casein to whey is about 8:2.

Another component of the methods and compositions of the present invention is one or more lipids. As used herein, the term “lipid” or “lipids” refers to fatty or oily organic compounds. The term “lipid” encompasses all fatty acid-type compounds. Lipids can be classified as either saturated, polyunsaturated, or monounsaturated.

Sources of lipids that are suitable for use with the methods and compositions of the present invention include, but are not limited to, coconut oil, fish oil (e.g., menhaden oil, sardine oil), nut oils (e.g., hazelnut oil, walnut oil, brazil nut oil, chestnut oil, peanut oil), pumpkin seed oil, rice bran oil, sesame seed oil, soy oil, soya oil, corn oil, safflower oil, evening primrose oil, rapeseed oil, olive oil, flaxseed (linseed) oil, cottonseed oil, high oleic safflower oil, palm stearin, soy lecithin, macadamia oil, MCT oil (medium chain triglyceride), sunflower oil, high oleic sunflower oil, palm oil, palm olein, palm kernel oil, canola oil, dairy fat (e.g., milk fat, butter, butter fat), wheat germ oil, whole grain oil, and mixtures thereof.

In some embodiments of the present invention, the lipid component comprises a blend of several different types of lipids. For example, the nutritional supplement may comprise a blend of at least one source of linoleic acid (an ω-3 fatty acid) and at least one source of α-linolenic acid (an ω-6 fatty acid). At least one source of DHA (an ω-3 fatty acid) is also present in the nutritional supplement of the present invention.

In certain aspects of the present invention, the types of lipids and the amount of lipids in the blend may be chosen so that the nutritional supplement has an ω-6:ω-3 fatty acid ratio of about 6:1 or less, about 5:1 or less, about 4:1 or less, about 3:1 or less, about 2:1 or less, or about 1:1 or less. In some embodiments, the nutritional supplement may simply have an ω-6:ω-3 fatty acid ratio of about 6:1 or less. In further embodiments, the nutritional supplement may have an ω-6:ω-3 fatty acid ratio somewhere in the range of between about 1:1 and 6:1, or between about 3:1 and about 6:1. In even further embodiments, the nutritional supplement may have an ω-6:ω-3 fatty acid ratio within the range of between about 5:1 and about 6:1. In still other embodiments, the nutritional supplement may have an ω-6:ω-3 fatty acid ratio of about 6:1. In still further embodiments, the nutritional supplement may have an ω-6:ω-3 fatty acid ratio of about 5:1.

For example, in certain aspects of the present invention, the nutritional supplement comprises a blend of 1) at least one source of the ω-3 fatty acid, linoleic acid, 2) at least one source of the ω-6 fatty acid, a-linolenic acid, and 3) at least one source of the ω-3 fatty acid, DHA, wherein the ratio of the amount of total ω-6 fatty acid to the amount of total ω-3 fatty acid is about 6:1 or less.

Suitable sources of oils rich in linoleic acid include, but are not limited to, sunflower oil, cottonseed oil, walnut oil, soybean oil, whole grain oil, wheat germ oil, chestnut oil, pumpkin seed oil, sesame oil, brazilnut oil, peanut oil, safflower oil, corn oil, animal fat, and mixtures thereof.

Suitable sources of oils rich in a-linolenic acid include, but are not limited to, linseed oil (or flaxseed oil), canola oil, soybean oil, walnut oil, wheat germ oil, and mixtures thereof.

Suitable sources of DHA include, but are not limited to, dairy products like eggs (e.g., egg yolk oil) and butterfat; marine or fish oils, such as cod, salmon, menhaden, sardine, tuna and many other fish; brain lipids, animal fats (e.g., beef and chicken fat), animal organs, lard, tallow; and microbial oils such as fungal and algal oils described in detail in U.S. Pat. Nos. 5,347,657; 5,550,156; and 5,658,767. Each of the aforesaid U.S. patents is incorporated herein in its entirety by reference thereto.

DHA that is suitable for use with the present invention may be isolated from any single-celled organism that contains a significant amount of DHA. This would include various oleaginous fungi, various algae (especially members of the class Dinophyceae, Bacillariophyceae, Chlorophyceae, Prymnesiophyceae and Euglenophyceae), as well as organisms of uncertain taxonomic status such as Thraustochytrium or Schizochytrium. Suitable processes for producing DHA-containing lipids from Thraustochytrium or Schizochytrium are provided in U.S. Pat. No. 5,130,242, which is incorporated herein in its entirety by reference thereto.

Algal oils such as those from dinoflagellates of the class Dinophyceae, notably Crypthecodinium cohnii may be suitable sources of DHA (including DHASCO™ and DHASCO-S™), as taught in U.S. Pat. Nos. 5,397,591; 5,407,957; 5,492,938; and 5,711,983, all of which are incorporated herein in their entireties by reference thereto. For example, Crypthecodinium cohnii has been manipulated to produce very high levels of DHA. This organism may be cultivated at a large scale and the biomass can be used for the production of a DHA-containing oil. Suitable processes for producing a DHA-containing biomass from C. cohnii are provided in U.S. Pat. Nos. 5,397,591 and 5,492,938, each of which is incorporated herein in its entirety by reference thereto.

The nutritional supplement of the present invention also encompasses any new source of DHA that may be developed synthetically or through genetic manipulation of organisms, such as vegetables and/or oil bearing plants. For example, desaturase and elongase genes have been identified from many organisms and these could be engineered into plant or other host cells to cause them to produce large quantities of DHA-containing lipids at a low cost. Therefore, the use of such synthetic or recombinant DHA-containing lipids is contemplated in the present invention.

In certain aspects of the present invention, the nutritional supplement comprises a blend of sources of lipids comprising two or more of canola oil, soybean oil, high oleic sunflower (HoSun) oil, medium chain triglyceride (MCT) oil, corn oil, and in certain aspects of the present invention, the blend comprises a blend of all five of these oils. In other aspects, the blend of sources of lipids comprises a blend of canola oil, soybean oil, high oleic sunflower oil, medium chain triglyceride (MCT) oil, and corn oil; and in combination with DHA oil, provides the nutritional supplement with an ω-6:ω-3 fatty acid ratio of about 6:1 or less.

In certain aspects of the present invention, the nutritional supplement may also contain other ingredients in addition to the carbohydrates, proteins, lipids, and DHA described previously, such as one or more vitamins, minerals, antioxidants, fibers, flavoring agents, coloring agents, preservatives, emulsifiers, and other dietary supplements, and mixtures thereof.

The methods and compositions of the present invention may optionally include one or more of the following vitamins or derivatives thereof, including, but not limited to, biotin, vitamin B₁, thiamin, thiamin pyrophosphate, vitamin B₂, riboflavin, flavin mononucleoride, flavin adenine dinucleotide, pyridoxine hydrochloride, thiamin mononitrate, folic acid, vitamin B₃, niacin, nicotinic acid, nicotinamide, niacinamide, nicotinamide adenine dinucleotide, tryptophan, biotin, pantothenic acid, vitamin B₆, vitamin B₁₂, cobalamin, methylcobalamin, deoxyadenosylcobalamin, cyanocobalamin, calcium pantothenate, pantothenic acid, vitamin C, ascorbic acid, vitamin A, retinol, retinal, retinoic acid, beta-carotene, vitamin D, vitamin D₃, calciferol, cholecalciferol, dihydroxy vitamin D, 1,25-dihydroxycholecalciferol, 7-dehyrdocholesterol, choline, vitamin E, vitamin E acetate, vitamin K, menadione, menaquinone, phylloquinone, naphthoquinone, and mixtures thereof.

The methods and compositions of the present invention may optionally include one or more of the following minerals or derivatives thereof, including, but not limited to, phosphorus, potassium, sulfur, sodium, docusate sodium, chloride, manganese, magnesium, magnesium stearate, magnesium carbonate, magnesium oxide, magnesium hydroxide, magnesium sulfate, copper, cupric sulfate, iodide, boron, zinc, zinc oxide, chromium, molybdenum, iron, carbonyl iron, ferric iron, ferrous fumarate, polysaccharide iron, fluoride, selenium, molybdenum, calcium phosphate or acetate, potassium phosphate, magnesium sulfate or oxide, sodium chloride, potassium chloride or acetate, ferric orthophosphate, alpha-tocopheryl acetate, zinc sulfate or oxide, copper gluconate, chromium chloride or picolonate, potassium iodide, sodium selenate, sodium molybdate, phylloquinone, cyanocobalamin, sodium selenite, copper sulfate, inositol, potassium iodide, cobalt, and mixtures thereof. Non-limiting exemplary derivatives of mineral compounds include salts, alkaline salts, esters and chelates of any mineral compound.

The minerals can be added in the form of salts such as calcium phosphate, calcium glycerol phosphate, sodium citrate, potassium chloride, potassium phosphate, magnesium phosphate, ferrous sulfate, zinc sulfate, cupric sulfate, manganese sulfate, and sodium selenite. Additional vitamins and minerals can be added as known within the art.

The methods and compositions of the present invention may optionally include one or more of the following flavoring agents, including, but not limited to, flavored extracts, volatile oils, cocoa or chocolate flavorings, peanut butter flavoring, cookie crumbs, vanilla or any commercially available flavoring. Examples of useful flavorings include, but are not limited to, pure anise extract, imitation banana extract, imitation cherry extract, chocolate extract, pure lemon extract, pure orange extract, pure peppermint extract, imitation pineapple extract, imitation rum extract, imitation strawberry extract, or vanilla extract; or volatile oils, such as balm oil, bay oil, bergamot oil, cedarwood oil, cherry oil, cinnamon oil, clove oil, or peppermint oil; peanut butter, chocolate flavoring, vanilla cookie crumb, butterscotch, toffee, and mixtures thereof. The amounts of flavoring agent can vary greatly depending upon the flavoring agent used. The type and amount of flavoring agent can be selected as is known in the art.

The methods and compositions of the present invention may optionally include one or more emulsifiers that may be added for stability of the final product. Examples of suitable emulsifiers include, but are not limited to, lecithin (e.g., from egg or soy), and/or mono- and di-glycerides, and mixtures thereof. Other emulsifiers are readily apparent to the skilled artisan and selection of suitable emulsifier(s) will depend, in part, upon the formulation and final product.

The methods and compositions of the present invention may optionally include one or more preservatives that may also be added to the nutritional supplement to extend product shelf life. Suitable preservatives include, but are not limited to, potassium sorbate, sodium sorbate, potassium benzoate, sodium benzoate, and calcium disodium EDTA, and mixtures thereof.

The methods and compositions of the present invention may optionally include one or more sources of fiber, including, but not limited to, soy fiber, pea hull fiber, soy hull fiber, gum acacia, and mixtures thereof. The nutritional supplement of the present invention encompasses any source of fibers that may be naturally-occurring, synthetic, or developed through the genetic manipulation of organisms, whether such new source is now known or later developed.

The methods and compositions of the present invention may optionally include one or more stabilizers. Suitable stabilizers for use in the methods and compositions of the present invention, include, but are not limited to, gum arabic, gum ghatti, gum karaya, gum tragacanth, agar, furcellaran, guar gum, gellan gum, locust bean gum, pectin, low methoxyl pectin, gelatin, microcrystalline cellulose, CMC (sodium carboxymethylcellulose), methylcellulose hydroxypropyl methyl cellulose, hydroxypropyl cellulose, DATEM (diacetyl tartaric acid esters of mono- and diglycerides), dextran, carrageenans, and mixtures thereof.

Except for the amounts of ω-3 and ω-6 fatty acids, the various amounts of the aforementioned ingredients that may be added to the nutritional supplements of the present invention can be chosen as is known in the art.

For example, carbohydrates are present in the nutritional supplements of the present invention in an amount that can range from about 1 gram (g)/100 kilocalories (kcal) to about 50 g/100 kcal. In other embodiments, the amount of carbohydrate can range from about 5 g/100 kcal to about 25 g/100 kcal. In still other embodiments, the amount of carbohydrate can range from about 10 g/100 kcal to about 15 g/100 kcal. In some embodiments, the amount of carbohydrate that is present in the nutritional supplements of the present invention can be about 12.8 g/100 kcal.

Proteins are present in the nutritional supplements of the present invention in an amount that can range from about 0.1 g/100 kcal to about 20 g/100 kcal. In other embodiments, the amount of protein can range from about 0.5 g/100 kcal to about 10 g/100 kcal. In still other embodiments, the amount of protein can range from about 1 g/100 kcal to about 5 g/100 kcal. In some embodiments, the amount of protein that is present in the nutritional supplements of the present invention can be about 2.8 g/100 kcal.

Lipids are present in the nutritional supplements of the present invention in an amount that can range from about 0.1 g/100 kcal to about 50 g/100 kcal. In other embodiments, the amount of lipid can range from about 1 g/100 kcal to about 20 g/100 kcal. In still other embodiments, the amount of lipid can range from about 2 g/100 kcal to about 10 g/100 kcal. In some embodiments, the amount of lipid that is present in the nutritional supplements of the present invention can be about 4.2 g/100 kcal.

When the source of lipids comprises linoleic acid, the amount of linoleic acid present in the nutritional supplements of the present invention can range from about 0.1 g/100 kcal to about 20 g/100 kcal. In other embodiments, the amount of linoleic acid can range from about 0.5 g/100 kcal to about 5 g/100 kcal or the amount can range from about 0.75 g/100 kcal to about 2 g/100 kcal. In still other embodiments, the amount of linoleic acid can be about 1 g/100 kcal.

When the source of lipids comprises a-linolenic acid, the amount of α-linolenic acid present in the nutritional supplements of the present invention can range from about 0.01 g/100 kcal to about 10 g/100 kcal. In other embodiments, the amount of α-linolenic acid can range from about 0.05 g/100 kcal to about 1 g/100 kcal or the amount can range from about 0.1 g/100 kcal to about 0.2 g/100 kcal. In still other embodiments, the amount of α-linolenic acid can be about 0.16 g/100 kcal.

The amount of DHA present in the nutritional supplements of the present invention can range from 0.1 milligram (mg)/100 kcal to about 1 g/100 kcal. In other embodiments, the amount of DHA can range from about 1 mg/100 kcal to about 200 mg/100 kcal or the amount can range from about 5 mg/100 kcal to about 50 mg/100 kcal. In still other embodiments, the amount of DHA can range from about 7.5 mg/100 kcal to about 25 mg/100 kcal. In certain aspects of the present invention, the amount of DHA can be about 17 mg/100 kcal. The amount of DHA present in the nutritional supplements of the present invention may vary within a wide range based on the caloric content of the supplement.

In some embodiments, the amount of DHA present in the nutritional supplements and methods described herein may comprise a meaningful level of DHA. As used herein, the terms “meaningful level” or “meaningful levels” refer to an amount of DHA that can provide a health benefit to at least one of the subjects described herein no matter how slight the benefit. Such health benefits may include, but are not limited to, increased dietary intakes, enhanced physical growth, improved brain and/or ocular development, improved immune health ad function, and improved cardiovascular health and function.

In other aspects of the present invention, the nutritional supplement may comprise a blend of sources of lipids comprising in % w/w of the total lipid component of between about 10% and about 50% canola oil, between about 5% and about 40% soybean oil, between about 5% and about 40% high oleic sunflower oil, between about 5% and about 40% medium chain triglyceride (MCT) oil, between about 1% and about 20% corn oil, and between about 0.1% and about 10% of a source of DHA. In one embodiment, the aforementioned nutritional supplement has an ω-6:ω-3 fatty acid ratio of about 6:1 or less.

The term “weight percentages” and the acronym “wt. %” or “w/w” as used herein refer to weight percentages based on the total weight of either one component of the nutritional supplement, which will be specified, or it will based on the nutritional supplement in its final form with all ingredients added.

In still other aspects of the present invention, the nutritional supplement of the present invention may comprise a blend of sources of lipids comprising in % w/w of the total lipid component of about 29.3% canola oil, about 20.7% soy oil, about 20.5% high oleic sunflower oil, about 19.8% medium chain triglyceride oil, about 8.8% corn oil, and about 0.9% of a source of DHA. In another embodiment, the aforementioned nutritional supplement has an ω-6:ω-3 fatty acid ratio of about 6:1 or less.

When vitamin A is present in the methods and/or compositions of the present invention, it is advantageously present in a range of about 1 mcg/100 kcal to about 500 mcg/100 kcal. More advantageously, the vitamin A can be present in the methods and/or compositions of the present invention in an amount of about 70 mcg/100 kcal.

When vitamin D is present in the methods and/or compositions of the present invention, it is advantageously present in a range of about 0.1 mcg/100 kcal to about 100 mcg/100 kcal. More advantageously, the vitamin D can be present in the methods and/or compositions of the present invention in an amount of about 1.24 mcg/100 kcal.

When vitamin E is present in the methods and/or compositions of the present invention, it is advantageously present in a range of about 0.1 mg to about 100 mg. More advantageously, the vitamin E can be present in the methods and/or compositions of the present invention in an amount of about 2.36 mg.

When vitamin K is present in the methods and/or compositions of the present invention, it is advantageously present in a range of about 0.1 mcg/100 kcal to about 100 mcg/100 kcal. More advantageously, the vitamin K can be present in the methods and/or compositions of the present invention in an amount of about 5.4 mcg/100 kcal.

When vitamin B₁ is present in the methods and/or compositions of the present invention, it is advantageously present in a range of about 0.01 mg/100 kcal to about 10 mg/100 kcal. More advantageously, the vitamin B₁ can be present in the methods and/or compositions of the present invention in an amount of about 0.16 mg/100 kcal.

When vitamin B₂ is present in the methods and/or compositions of the present invention, it is advantageously present in a range of about 0.01 mg/100 kcal to about 10 mg/100 kcal. More advantageously, the vitamin B₂ can be present in the methods and/or compositions of the present invention in an amount of about 0.2 mg/100 kcal.

When vitamin B₆ is present in the methods and/or compositions of the present invention, it is advantageously present in a range of about 0.01 mg/100 kcal to about 10 mg/100 kcal. More advantageously, the vitamin B₆ can be present in the methods and/or compositions of the present invention in an amount of about 0.2 mg/100 kcal.

When vitamin B₁₂ is present in the methods and/or compositions of the present invention, it is advantageously present in a range of about 0.01 mcg/100 kcal to about 10 mcg/100 kcal. More advantageously, the vitamin B₁₂ can be present in the methods and/or compositions of the present invention in an amount of about 0.56 mcg/100 kcal.

When niacin is present in the methods and/or compositions of the present invention, it is advantageously present in a range of about 0.1 mg/100 kcal to about 100 mg/100 kcal. More advantageously, the niacin can be present in the methods and/or compositions of the present invention in an amount of about 2.45 mg/100 kcal.

When folic acid is present in the methods and/or compositions of the present invention, it is advantageously present in a range of about 0.1 mcg/100 kcal to about 500 mcg/100 kcal. More advantageously, the folic acid can be present in the methods and/or compositions of the present invention in an amount of about 20 mcg/100 kcal.

When pantothenic acid is present in the methods and/or compositions of the present invention, it is advantageously present in a range of about 0.1 mg/100 kcal to about 100 mg/100 kcal. More advantageously, the pantothenic acid can be present in the methods and/or compositions of the present invention in an amount of about 1.24 mg/100 kcal.

When biotin is present in the methods and/or compositions of the present invention, it is advantageously present in a range of about 0.1 mcg/100 kcal to about 100 mcg/100 kcal. More advantageously, the biotin can be present in the methods and/or compositions of the present invention in an amount of about 15.2 mcg/100 kcal.

When vitamin C is present in the methods and/or compositions of the present invention, it is advantageously present in a range of about 0.01 mg/100 kcal to about 1 g/100 kcal. More advantageously, the vitamin C can be present in the methods and/or compositions of the present invention in an amount of about 23.2 mg/100 kcal.

When choline is present in the methods and/or compositions of the present invention, it is advantageously present in a range of about 0.1 mg/100 kcal to about 500 mg/100 kcal. More advantageously, the choline can be present in the methods and/or compositions of the present invention in an amount of about 25.2 mg/100 kcal.

When inositol is present in the methods and/or compositions of the present invention, it is advantageously present in a range of about 0.1 mg/100 kcal to about 500 mg/100 kcal. More advantageously, the inositol can be present in the methods and/or compositions of the present invention in an amount of about 6.8 mg/100 kcal.

When taurine is present in the methods and/or compositions of the present invention, it is advantageously present in a range of about 0.1 mg/100 kcal to about 500 mg/100 kcal. More advantageously, the taurine can be present in the methods and/or compositions of the present invention in an amount of about 6 mg/100 kcal.

When carnitine is present in the methods and/or compositions of the present invention, it is advantageously present in a range of about 0.1 mg/100 kcal to about 500 mg/100 kcal. More advantageously, the carnitine can be present in the methods and/or compositions of the present invention in an amount of about 6 mg/100 kcal.

When calcium is present in the methods and/or compositions of the present invention, it is advantageously present in a range of about 0.1 mg/100 kcal to about 1 g/100 kcal. More advantageously, the calcium can be present in the methods and/or compositions of the present invention in an amount of about 96 mg/100 kcal.

When phosphorus is present in the methods and/or compositions of the present invention, it is advantageously present in a range of about 0.1 mg/100 kcal to about 1 g/100 kcal. More advantageously, the phosphorus can be present in the methods and/or compositions of the present invention in an amount of about 80 mg/100 kcal.

When magnesium is present in the methods and/or compositions of the present invention, it is advantageously present in a range of about 0.1 mg/100 kcal to about 500 mg/100 kcal. More advantageously, the magnesium can be present in the methods and/or compositions of the present invention in an amount of about 14 mg/100 kcal.

When iron is present in the methods and/or compositions of the present invention, it is advantageously present in a range of about 0.1 mg/100 kcal to about 100 mg/100 kcal. More advantageously, the iron can be present in the methods and/or compositions of the present invention in an amount of about 1 mg/100 kcal.

When zinc is present in the methods and/or compositions of the present invention, it is advantageously present in a range of about 0.1 mg/100 kcal to about 100 mg/100 kcal. More advantageously, the zinc can be present in the methods and/or compositions of the present invention in an amount of about 1 mg/100 kcal.

When manganese is present in the methods and/or compositions of the present invention, it is advantageously present in a range of about 0.01 mg/100 kcal to about 10 mg/100 kcal. More advantageously, the manganese can be present in the methods and/or compositions of the present invention in an amount of about 0.16 mg/100 kcal.

When copper is present in the methods and/or compositions of the present invention, it is advantageously present in a range of about 0.1 mcg/100 kcal to about 1 mg/100 kcal. More advantageously, the copper can be present in the methods and/or compositions of the present invention in an amount of about 80 mcg/100 kcal.

When iodine is present in the methods and/or compositions of the present invention, it is advantageously present in a range of about 0.1 mcg/100 kcal to about 100 mcg/100 kcal. More advantageously, the iodine can be present in the methods and/or compositions of the present invention in an amount of about 12 mcg/100 kcal.

When selenium is present in the methods and/or compositions of the present invention, it is advantageously present in a range of about 0.01 mcg/100 kcal to about 1 mg/100 kcal. More advantageously, the selenium can be present in the methods and/or compositions of the present invention in an amount of about 3 mcg/100 kcal.

When sodium is present in the methods and/or compositions of the present invention, it is advantageously present in a range of about 0.1 mg/100 kcal to about 500 mg/100 kcal. More advantageously, the sodium can be present in the methods and/or compositions of the present invention in an amount of between about 55 and 60 mg/100 kcal.

When potassium is present in the methods and/or compositions of the present invention, it is advantageously present in a range of about 0.1 mg/100 kcal to about 1 g/100 kcal. More advantageously, the potassium can be present in the methods and/or compositions of the present invention in an amount of about 180 mg/100 kcal.

When chloride is present in the methods and/or compositions of the present invention, it is advantageously present in a range of about 0.1 mg/100 kcal to about 1 g/100 kcal. More advantageously, the chloride can be present in the methods and/or compositions of the present invention in an amount of about 70 mg/100 kcal. The amount of sodium, potassium and chloride present in the nutritional supplements of the present invention may vary within a wide range based on the caloric content of the supplement.

When chromium is present in the methods and/or compositions of the present invention, it is advantageously present in a range of about 1 mcg/100 kcal to about 500 mcg/100 kcal.

When molybdenum is present in the methods and/or compositions of the present invention, it is advantageously present in a range of about 1 mcg/100 kcal to about 500 mcg/100 kcal.

When fluoride is present in the methods and/or compositions of the present invention, it is advantageously present in a range of about 1 mg/100 kcal to about 500 mg/100 kcal.

When a source of fiber is present in the methods and/or compositions of the present invention, the amount of fiber can range from about 0.01 g/100 kcal to about 100 g/100 kcal. In other embodiments, the amount of fiber can range from about 0.1 g/100 kcal to about 10 g/100 kcal or the amount can range from about 0.5 g/100 kcal to about 1 g/100 kcal. In still other embodiments, the amount of fiber can be about 0.6 g/100 kcal.

The nutritional supplement of the present invention may also contain one or more prebiotics. The term “prebiotic” means a non-digestible food ingredient that stimulates the growth and/or activity of probiotics. Any prebiotic known in the art will be acceptable in this embodiment. Prebiotics of the present invention may include lactulose, galacto-oligosaccharide, fructo-oligosaccharide, isomalto-oligosaccharide, soybean oligosaccharides, lactosucrose, xylo-oligosacchairde, inulin, polydextrose and gentio-oligosaccharides.

The nutritional supplement of the invention may also be administered to a subject in combination with human or animal milk (e.g., cow or goat milk).

In some embodiment, the liquid nutritional supplement contains about 250 kcal per serving. In other embodiments, the liquid nutritional supplement contains less than about 200 kcal per serving. In another embodiment, the liquid nutritional supplement contains between about 150 and 200 kcal per serving. In yet another embodiment, the liquid nutritional supplement contains between about 160 and 180 kcal per serving. In a further embodiment, the liquid nutritional supplement contains about 170 kcal per serving.

In some embodiments, the present invention is designed to deliver between about 7 and 20 mg/100 kcal DHA in a liquid nutritional supplement containing 250 kcal per serving. Thus, providing children between the ages of 1 and 10 with 2 to 4 servings of the supplement daily would result in from about 35 to about 200 mg supplemental DHA per day and would, therefore, address some of the gap between DHA intakes in this age group relative to the desired level of DHA consumption based on existing recommendations.

In other embodiments, the present invention is designed to deliver between about 7 and 75 mg/100 kcal DHA in a liquid nutritional supplement containing less than about 200 kcal per serving. In yet another embodiment, the present invention is designed to deliver between 7 and 75 mg/100 kcal DHA in a liquid nutritional supplement containing between about 150 and 200 kcal per serving. In a particular embodiment, the present invention is designed to deliver between 7 and 75 mg/100 kcal DHA in a liquid nutritional supplement containing between about 160 and 180 kcal per serving. In another embodiment, the present invention is designed to deliver between 7 and 75 mg/100 kcal DHA in a liquid nutritional supplement containing about 170 kcal per serving.

In certain otherembodiments, the liquid nutritional supplement contains less than about 200 kcal per serving and does not contain an artificial sweetener. In yet another embodiment, the liquid nutritional supplement contains between about 150 and 200 kcal per serving and does not contain an artificial sweetener. In yet another embodiment, the liquid nutritional supplement contains between about 160 and 180 kcal per serving and does not contain an artificial sweetener. In even another embodiment, the liquid nutritional supplement contains about 170 kcal per serving and does not contain an artificial sweetener.

The nutritional supplement of the invention may be provided in any form suitable for administration to a subject. For example, the nutritional supplement may be prepared in forms such as a liquid, powder, liquid/powder concentrate, ready-to-feed form, caplets, tablets, pills, capsules, puddings, chewable tablets, quick dissolve tablets, effervescent tablets, reconstitutable powders, elixirs, solutions, suspensions, emulsions, multi-layer tablets, bi-layer tablets, soft gelatin capsules, hard gelatin capsules, lozenges, chewable lozenges, beads, granules, particles, microparticles, dispersible granules, cachets, implants, depot implants, ingestibles, injectables, infusions, health bars, cookies, confections, animal feeds, cereals, cereal coatings, foods, soups and soup concentrates, nutritive foods, functional foods, and combinations thereof.

However, in certain aspects of the present invention, the nutritional supplement is provided in a liquid form. The preparation of the above dosage forms can be conducted as is known in the art.

The nutritional supplements of this invention can be manufactured using techniques well known to those skilled in the art. For example, various processing techniques exist for producing powdered, ready-to-feed and liquid supplements.

Typically, these techniques include formation of a slurry from one or more solutions which may contain water and one or more of the following: carbohydrates, proteins, lipids, stabilizers, vitamins and minerals. The pH of the slurry can be checked and, if required, adjusted to a relatively neutral pH, i.e., from about 6.5 to about 7.5 preferably about 7.0. The pH can be adjusted as required using any acid or base compatible with the present compositions, preferably citric acid, potassium hydroxide, or sodium phosphate dibasic.

This slurry is then emulsified, homogenized and cooled. Various other solutions may be added to the slurry before processing, after processing or at both times. In certain aspects of the present invention, the liquid nutritional supplement is sterile. In those embodiments, the supplement is aseptically sterilized and may be diluted to be utilized on a ready-to-feed basis or stored in a liquid or a powder. Homogenization and sterilization can be accomplished using standard equipment known to skilled artisans.

If the resulting nutritional supplement is meant to be a ready-to-feed liquid or concentrated liquid, an appropriate amount of water could be added before sterilization. If the resulting nutritional supplement is meant to be a powder, the slurry will be heated and dried to obtain a powder. The powder resulting from drying may be dry blended with further ingredients, if desired. The nutritional supplement can be packaged for distribution and sale as desired using standard equipment known in the art.

Variations on any of these manufacturing processes are known to or will be readily apparent to those skilled in the art. It is not intended that the invention be limited to any particular process of manufacture.

The following examples describe various embodiments of the present invention. Other embodiments within the scope of the claims herein will be apparent to one skilled in the art from consideration of the specification or practice of the invention as disclosed herein. It is intended that the specification, together with the examples, be considered to be exemplary only, with the scope and spirit of the invention being indicated by the claims which follow the examples. In the examples, all percentages are given on a weight basis unless otherwise indicated.

### EXAMPLE 1

This example illustrates one embodiment of the liquid nutritional supplement composition of the present invention. Table 1 illustrates the ingredients present in an embodiment of the present liquid nutritional supplement and their amounts in grams (g) or milligrams (mg), expressed per Liter (L). Table 2 illustrates the amount of base nutrients (proteins, fats and carbohydrates), as well as vitamins and mineral nutrients present in 8 fl. oz. of an embodiment of the present liquid nutritional supplement. For purposes of the present composition, the caloric content in 8 fl. oz. of an embodiment of the present liquid nutritional supplement is approximately 250 kcal.

TABLE 1
Ingredient Information and Concentrations (Per Liter)

| Ingredient, Unit | Per Liter |
|---|---|
| Sucrose, g | 115.27 |
| Maltodextrin, DE15, g | 55.00 |
| Milk Protein Concentrate, g | 35.00 |
| Nutritional Oil Blend, g (canola oil, | 25.50 |
| corn oil and high oleic sunflower oil) | |
| Soybean oil, g | 9.00 |
| MCT oil, g | 8.65 |
| Natural and Artificial Flavor, g | 3.83 |
| Potassium Citrate, g | 2.00 |
| Sodium Ascorbate, mg | 719.60 |
| Corn Syrup Solids, mg | 404.28 |
| Inositol, mg | 104.72 |
| Taurine, mg | 76.16 |
| Niacinamide, mg | 29.68 |
| Biotin Trituration, mg | 22.82 |
| Calcium Pantothenate, mg | 22.12 |
| Vitamin B₁₂, mg | 9.48 |
| Pyridoxine Hydrochloride, mg | 4.34 |
| Thiamin Hydrochloride, mg | 3.25 |
| Riboflavin, mg | 3.08 |
| Folic Acid, mg | 0.28 |
| Potassium Iodide, mg | 0.20 |
| Potassium Phosphate, Dibasic, mg | 1300.00 |
| Magnesium Chloride, mg | 1280.00 |
| Calcium Phosphate, Tribasic, mg | 1250.00 |
| Sodium Chloride, mg | 540.00 |
| Lecithin, mg | 440.00 |
| Single Cell DHA oil, mg | 430.00 |
| Choline Chloride, mg | 400.00 |
| Carrageenan K-100, mg | 125.00 |
| Tocopheryl Acetate, mg | 48.65 |
| Canola oil, mg | 36.56 |
| Vitamin A palmitate, mg | 1.35 |
| Cholecalciferol Concentrate, mg | 0.83 |
| Vitamin K₁, Liquid Phytonadione, mg | 0.11 |
| Carnitine, mg | 80.00 |
| Ferrous sulfate, mg | 63.00 |
| Zinc Sulfate, Monohydrate, mg | 33.51 |
| Corn Syrup Solids, mg | 11.52 |
| Manganese Sulfate, Monohydrate, mg | 5.63 |
| Cupric Sulfate, mg | 4.79 |
| Chromic Chloride Hexahydrate, mg | 0.31 |
| Sodium Molybdate Dihydrate, mg | 0.15 |
| Sodium Selenite, mg | 0.08 |

TABLE 2
Nutrient Information

| Nutrient, Unit | Per 8 fl. oz. |
|---|---|
| Protein, g | 7.1 |
| Fat, g | 10.5 |
| Saturated Fat, g | 3 |
| Cholesterol, mg | <5 |
| Linoleic Acid, g | 2.5 |
| alpha-Linolenic Acid, g | 0.4 |
| DHA, mg | 43 |
| Carbohydrates, g | 32 |
| Vitamin A, IU | 420 |
| Vitamin D, IU | 125 |
| Vitamin E, IU | 8.8 |
| Vitamin K, mcg | 14 |
| Thiamin (Vitamin B₁), mg | 0.4 |
| Riboflavin (Vitamin B₂), mg | 0.5 |
| Pyridoxine, mg | 0.5 |
| Vitamin B₁₂, mcg | 1.4 |
| Niacin, mg | 4.9 |
| Folic Acid, mcg | 50 |
| Pantothenic Acid, mg | 3.1 |
| Biotin, mcg | 38 |
| Choline, mg | 63 |
| Vitamin C, mg | 58 |
| Calcium, mg | 240 |
| Phosphorus, mg | 200 |
| Magnesium, mg | 40 |
| Sodium, mg | 88 |
| Potassium, mg | 310 |
| Chloride, mg | 175 |
| Iodine, mcg | 30 |
| Iron, mg | 2.5 |
| Zinc, mg | 2.5 |
| Manganese, mg | 0.4 |
| Copper, mg | 0.2 |
| Selenium, mcg | 7.5 |
| Chromium, mcg | 12.5 |
| Molybdenum, mcg | 12.5 |
| Taurine, mg | 15 |
| L-carnitine, mg | 15 |
| Inositol, mg | 20 |

Table 3 illustrates the concentration of relevant components in the nutritional supplement of Example 1 expressed in grams per liter.

TABLE 3
Component Concentrations (Per Liter)

| Component, Unit | Per Liter |
|---|---|
| Protein, g | 30 |
| Carbohydrate, g | 135 |
| Lipids, g | 44 |
| Linoleic acid, g | 10.6 |
| α-Linolenic acid, g | 1.7 |
| Docosahexaenoic acid, g | 0.18 |

The caloric distribution of the nutritional supplement of Example 1 is depicted in Table 4.

TABLE 4
Caloric Distribution

| Component | Caloric Percentages |
|---|---|
| Protein | 11% |
| Lipid | 38% |
| Carbohydrate | 51% |

The following Table 5 describes the content of the LCPUFAs in the nutritional supplement of Example 1.

TABLE 5
LCPUFA Information

| LCPUFA | % w/w of total lipid | % of total calories |
|---|---|---|
| Linoleic acid (LA) | 24.1% | 9.06% |
| α-Linolenic acid (ALA) | 3.9% | 1.47% |
| Docosahexaenoic acid (DHA) | 0.41% | 0.154% |

### EXAMPLE 2

This example illustrates another embodiment of the liquid nutritional supplement composition of the present invention. Table 6 illustrates the ingredients present in an embodiment of the present liquid nutritional supplement and their amounts expressed on a weight percentage of the total weight of the nutritional supplement. Table 7 illustrates the amount of base nutrients (proteins, fats and carbohydrates), as well as vitamins and mineral nutrients present in 8 fl. oz. of an embodiment of the present liquid nutritional supplement. In this example, the caloric content in 8 fl. oz. of an embodiment of the present liquid nutritional supplement is approximately 170 kcal.

TABLE 6
Ingredient Information and Concentrations (Per Liter)

| Ingredient, g | Per Liter |
|---|---|
| Skim Milk, Condensed | 170 |
| Sucrose | 42.0 |
| Fructose, Crystalline | 35.0 |
| Nutritional Fat Blend (canola oil, corn | 31.3 |
| oil and high oleic sunflower oil) | |
| Cocoa Powder | 9.0 |
| Whey Protein Concentrate | 5.0 |
| Sodium Caseinate | 3.0 |
| Single Cell DHA Oil | 1.45 |
| Calcium Phosphate, Tribasic | 1.10 |
| Natural and Artificial Flavor | 0.50 |
| Lecithin Concentrate | 0.50 |
| Guar Gum | 0.35 |
| Mono- and Diglycerides | 0.30 |
| Sodium Ascorbate | 0.15 |
| Corn Syrup Solids | 0.08 |
| Niacinamide | 0.03 |
| Biotin Trituration | 0.02 |
| Calcium Pantothenate | 0.004 |
| Pyridoxine Hydrochloride | 0.002 |
| Thiamine Hydrochloride | 0.002 |
| Riboflavin | 0.0007 |
| Folic Acid | 0.0003 |
| Carrageenan K-100 | 0.25 |
| Sodium Chloride | 0.25 |
| Magnesium Phosphate, Dibasic | 0.15 |
| Zinc Sulfate | 0.02 |
| Manganese Sulfate, Monohydrate | 0.007 |
| Cupric Sulfate | 0.005 |
| Corn Syrup Solids | 0.002 |
| Ferric Pyrophosphate | 0.03 |
| Soybean Oil | 0.02 |
| Tocopheryl Acetate, DL-Alpha | 0.004 |
| Vitamin A Palmitate | 0.003 |
| Cholecalciferol Concentrate | 0.0002 |

TABLE 7
Nutrient Information

| Nutrient, Unit | Per 8 fl. oz. |
|---|---|
| Calories, kcal | 170 |
| Protein, g | 6 |
| Fat, g | 5 |
| Saturated Fat, g | 0.7 |
| Cholesterol, mg | 0 |
| Linoleic Acid, g | 1.0 |
| alpha-Linolenic Acid, g | 0.2 |
| DHA, mg | 130 |
| Carbohydrates, g | 25 |
| Vitamin A, IU | 820 |
| Vitamin D, IU | 48 |
| Vitamin E, IU | 2.7 |
| Thiamin (Vitamin B₁), mg | 0.32 |
| Riboflavin (Vitamin B₂), mg | 0.36 |
| Pyridoxine, mg | 0.36 |
| Vitamin B₁₂, mcg | 0.2 |
| Niacin, mg | 5.8 |
| Folic Acid, mcg | 70 |
| Pantothenic Acid, mg | 1.3 |
| Biotin, mcg | 30 |
| Vitamin C, mg | 10 |
| Calcium, mg | 270 |
| Phosphorus, mg | 210 |
| Magnesium, mg | 38 |
| Sodium, mg | 95 |
| Potassium, mg | 320 |
| Chloride, mg | 180 |
| Iodine, mcg | 70 |
| Iron, mg | 2.7 |
| Zinc, mg | 2.5 |
| Manganese, mg | 0.79 |
| Copper, mg | 0.3 |

Table 8 illustrates the concentration of relevant components in the nutritional supplement of Example 2 expressed in grams per liter.

TABLE 8
Component Concentrations (Per Liter)

| Component, Unit | Per Liter |
|---|---|
| Protein, g | 25.3 |
| Carbohydrate, g | 105 |
| Lipids, g | 21.1 |

The caloric distribution of the nutritional supplement of Example 2 is depicted in Table 9.

TABLE 9
Caloric Distribution

| Component | Caloric Percentages |
|---|---|
| Protein | 14% |
| Carbohydrate | 59% |
| Lipid | 27% |

### EXAMPLE 3

This example illustrates a procedure for manufacturing an embodiment of the nutritional supplement of the present invention.

The composition of an embodiment of the present invention can be found in the listing of components in Table 1. In that embodiment, the protein source comprises a milk protein concentrate, which is a blend of casein and whey protein at a ratio of 8:2. The lipid source comprises a blend of the following based on w/w of the total lipid content: 29.3% canola oil, 20.7% soy oil, 20.5% high oleic sunflower oil, 19.8% medium chain triglyceride oil, 8.8% corn oil, and 0.9% DHA oil. The composition has a DHA content of about 17 mg/100 kcal. The carbohydrate source comprises a blend of maltodextrin and sucrose. The composition also comprises a suitable amount of carrageenan and a flavoring agent.

A procedure that may be followed to manufacture the liquid nutritional supplement of Example 1 comprises the following steps:
1. The protein blend and the carbohydrate blend can be dispersed in water at room temperature with good mixing.
2. The pH of the dispersion can then be adjusted to about 7 with potassium hydroxide.
3. All vitamins and minerals can be dissolved in water, preheated at about 170° F., and the resulting solution can be added to the protein/carbohydrate dispersion.
4. The pH can then be readjusted to about 7.
5. The lipid blend can be preheated to about 160° F. and then added to the carbohydrate/protein/vitamin and mineral suspension with good mixing. Any optional ingredients can also be added at this time.
6. The mixture can then be heated with direct steam injection to 225° F. for 45 seconds, and then flash cooled to 160° F.
7. The mixture can then be homogenized twice using a two-stage homogenizer, at a total pressure of 3000 psig, with 2500 psig for the first stage and 500 psig for the second stage.
8. The mixture can then be aseptically sterilized at 268° F. for 5 seconds at 2000 psig pressure and then packaged using a Dole Aseptic canning unit, Model 1305.

All references cited in this specification, including without limitation, all papers, publications, patents, patent applications, presentations, texts, reports, manuscripts, brochures, books, internet postings, journal articles, periodicals, and the like, are hereby incorporated by reference into this specification in their entireties.

The discussion of the references herein is intended merely to summarize the assertions made by their authors and no admission is made that any reference constitutes prior art. Applicants reserve the right to challenge the accuracy and pertinency of the cited references.

Although preferred embodiments of the invention have been described using specific terms, devices, and methods, such description is for illustrative purposes only. The words used are words of description rather than of limitation. It is to be understood that changes and variations may be made by those of ordinary skill in the art without departing from the spirit or the scope of the present invention, which is set forth in the following claims. In addition, it should be understood that aspects of the various embodiments may be interchanged both in whole or in part.

What is claimed is:

1. A nutritional supplement for administration to children, the nutritional supplement comprising:
a protein component;
a carbohydrate component; and
a lipid component comprising a source of DHA, wherein the supplement has an ω-6:ω-3 fatty acid ratio of about 6:1 or less.

2. The nutritional supplement according to claim 1, wherein the nutritional supplement has an ω-6:ω-3 fatty acid ratio of about 3:1 or less.

3. The nutritional supplement according to claim 1, wherein the nutritional supplement has an ω-6:ω-3 fatty acid ratio of about 1:1.

4. The nutritional supplement according to claim 1, wherein the supplement is nutritionally complete.

5. The nutritional supplement according to claim 1, wherein the supplement is a liquid.

6. The nutritional supplement according to claim 1, wherein the carbohydrate component comprises a mixture of maltodextrin and sucrose.

7. The nutritional supplement according to claim 1, wherein the protein component comprises milk protein concentrate.

8. The nutritional supplement according to claim 1, wherein the lipid component comprises a mixture of at least one source of linoleic acid and at least one source of α-linolenic acid.

9. The nutritional supplement according to claim 1, wherein the lipid component comprises a mixture of canola oil, soybean oil, high oleic sunflower oil, medium chain triglyceride (MCT) oil, and corn oil.

10. The nutritional supplement according to claim 1, wherein the source of DHA comprises single-celled organisms.

11. The nutritional supplement according to claim 1, wherein the nutritional supplement contains DHA in an amount of between about 0.1 mg/100 kcal of supplement and about 1 g/100 kcal of supplement.

12. The nutritional supplement according to claim 1, wherein the nutritional supplement contains DHA in an amount of between about 5 mg/100 kcal of supplement and about 50 mg/100 kcal of supplement.

13. The nutritional supplement according to claim 1, wherein the nutritional supplement contains DHA in an amount of between about 7.5 mg/100 kcal of supplement and about 25 mg/100 kcal of supplement.

14. The nutritional supplement according to claim 1, wherein the nutritional supplement contains DHA in an amount of about 17 mg/100 kcal of supplement.

15. The nutritional supplement according to claim 1, further comprising human or animal milk.

16. The nutritional supplement according to claim 1, further comprising at least one prebiotic.

17. The nutritional supplement according to claim 24, wherein the prebiotic is selected from the group consisting of lactulose, galacto-oligosaccharide, fructo-oligosaccharide, isomalto-oligosaccharide, soybean oligosaccharides, lactosucrose, xylo-oligosacchairde, inulin, polydextrose and gentio-oligosaccharides.

18. The nutritional supplement according to claim 1, wherein the supplement contains about 250 kcal per serving.

19. The nutritional supplement according to claim 1, wherein the supplement contains between about 160 and 180 kcal per serving.

20. The nutritional supplement according to claim 20, wherein the supplement does not contain artificial sweeteners.

21. The nutritional supplement according to claim 20, wherein the sweeteners in the supplement consist of sucrose and fructose.

22. A nutritional supplement for administration to children, the nutritional supplement comprising:
a protein component;
a carbohydrate component; and
a lipid component comprising, in % w/w of the total lipid component:
between about 10% and about 50% canola oil;
between about 5% and about 40% soy oil;
between about 5% and about 40% high oleic sunflower oil;
between about 5% and about 40% medium chain triglyceride oil;
between about 1% and about 20% corn oil; and
between about 0.1% and about 10% of a source of DHA.

23. The nutritional supplement according to claim 23, wherein the lipid component comprises, in % w/w of the total lipid component:
29.3% canola oil;
20.7% soy oil;
20.5% high oleic sunflower oil;
19.8% medium chain triglyceride oil;
8.8% corn oil; and
0.9% of a source of DHA.

24. The nutritional supplement according to claim 23, wherein the supplement has an ω-6:ω-3 fatty acid ratio of about 6:1 or less.

25. A liquid pediatric nutritional supplement comprising:
a protein component that comprises milk protein concentrate;
a carbohydrate component that comprises a mixture of maltodextrin and sucrose;
a vitamin and mineral component; and
a lipid component comprising, in % w/w of the total lipid component, about:
29.3% canola oil;
20.7% soy oil;
20.5% high oleic sunflower oil;
19.8% medium chain triglyceride oil;
8.8% corn oil; and
0.9% of a source of DHA,
wherein the supplement is nutritionally complete and has an ω-6: ω-3 fatty acid ratio of about 6:1 or less.

26. A method of providing nutrition to a pediatric subject comprising administering to the subject a nutritional supplement comprising a protein component, a carbohydrate component, and a lipid component that comprises a source of DHA, wherein the nutritional supplement has an ω-6:ω-3 fatty acid ratio of about 6:1 or less.

27. The method according to claim 27, wherein the subject is between the age of about 9 months and about 13 years.

28. The method according to claim 27, wherein the subject is between the age of about 1 year and 10 years.

29. The method according to claim 34, wherein the subject suffers from chronic illness, injury, trauma, failure to thrive, slow growth, undernourishment, poor immune function, poor cardiovascular function, weak bone strength, poor mental health development, recovery from surgery or illness, poor appetite, anorexia, bulimia, impaired ability to digest other sources of nutrition, and mixtures thereof.

30. The method according to claim 34, wherein the supplement is administered to the subject in combination with human or animal milk.
